(19) United States
(12) Patent Application Publication
(10) Pub. No.: US 2008/0107711 A1
(43) Pub. Date: May 8, 2008

(54) Bioabsorbable Implantable Material Fortified with Antibiotics for Localized Delivery of Drugs

(76) Inventor: Alexis Paul Shelokov, Dallas, TX (US)

Correspondence Address: GARDERE WYNNE SEWELL LLP;INTELLECTUAL PROPERTY SECTION, 3000 THANKSGIVING TOWER, 1601 ELM ST, DALLAS, TX, 75201-4761, US

(21) Appl. No.: 11/557,773

(22) Filed: Nov. 8, 2006

(51) Int. Cl. A61K 9/00

(52) U.S. Cl. 424/426

(57) ABSTRACT

A bioabsorbable, implantable, flexible sheet of material contains a predetermined quantity of one or more selected antibiotics. The sheet of material is adapted to be placed inside a body for delivery of antibiotics. The sheet of material may be an implantable plastic or other suitable implantable material. The antibiotics may be in a crystalline or non-crystalline form. The sheet of material can be wrapped about an implant, a bone, a tissue or any other part inside a body.

### FIELD OF THE INVENTION

The present invention relates to antibiotics delivery systems and methods. More specifically, the present invention relates to bioabsorbable, implantable material fortified with antibiotics for localized delivery of drugs.

### BACKGROUND OF THE INVENTION

Various implants are used in spinal and other surgeries. For example, disks, plates, screws, artificial joints, and femoral and tibial rods are used as orthopedic implants. During perioperative periods, infections often develop in orthopedic implants. Specifically, in metallic implants, infection may develop due to contamination by Staph species.

Surgeons generally try to reduce the risk of infection by intra venus (IV) administration of antibiotics. Antibiotics, however, do not easily penetrate hard tissues and bony environments where these infections typically occur. Consequently, surgeons often face difficulty in sterilizing these infections.

In order to more effectively treat infections related to orthopedic implants and to reduce the risk of future infections, it is preferable to deliver antibiotics locally. Antibiotics may be delivered locally by the inflow and outflow irrigation method. As will be appreciated by those skilled in the art, in the inflow and outflow irrigation method, tubes are implanted under the skin to deliver antibiotics containing fluid to the wound. Antibiotics containing fluid is dripped into the wound. An egress is provided for the fluid to drain out. The inflow and outflow irrigation method has proven to have limited effectiveness. The method is often unreliable and difficult to maintain at a steady state. Also, its effectiveness in delivering high local concentration of antibiotics is yet to be proven.

Antibiotics may also be delivered locally by applying bone cement containing antibiotics in the wound. For example, antibiotics may be added to bone cement, which is then implanted in the patient during a hip replacement surgery. The antibiotics in the bone cement elute into the wound decreasing the infection rate.

If perioperative infection related to orthopedic implants develops, a patient must undergo a subsequent operation to remove the implants and to sterilize the infection. As a result of the subsequent operation, the total cost of treatment related to orthopedic implants increases dramatically.

Accordingly, there exists a need to reduce the risk of implant-related infections. There exists a need to for new methods to deliver antibiotics locally to the wound. There exists a need to reduce the likelihood of subsequent surgeries to treat infections due to implants and thus prevent the increase in cost.

### SUMMARY OF THE INVENTION

The present invention is directed to a bioabsorbable, implantable material that contains one or more selected antibiotics. The bioabsorbable, implantable material may be a bioabsorbable, implantable plastic impregnated with one or more antibiotics. The bioabsorbable, implantable material (for example, plastic) may be impregnated with one or more antibiotics and rolled into a sheet, a cellophane or a film. A flexible laminate of bioabsorbable, implantable sheet or cellophane may be formed by bonding a plurality of layers of bioabsorbable, implantable materials. The individual layers may be impregnated with one or more antibiotics. Individual layers may have different antibiotics. Also, individual layers may have different absorption characteristics, thus allowing a controlled release of drugs over a predetermined period of time.

The bioabsorbable, implantable material impregnated with antibiotics can also be formed into crumbs and implanted in a body for localized delivery of drugs. Also, the bioabsorbable, implantable material impregnated with antibiotics can be formed into various structures for use as implants.

The bioabsorbable, implantable sheets, cellophanes or films are adapted to be placed inside a body for delivery of antibiotics. For example, the sheets, cellophanes or films may be wrapped around an orthopedic implant for localized delivery of antibiotics. The antibiotics may be in a crystalline or non-crystalline form.

### BRIEF DESCRIPTION OF THE DRAWINGS

For a more complete understanding of the features and advantages of the present invention, reference is now made to the detailed description of the invention along with the accompanying figures and in which:

FIG. 1 illustrates a sheet of bioabsorbable, implantable material impregnated with antibiotics.

FIG. 2 illustrates a laminate of bioabsorbable, implantable material impregnated with antibiotics.

FIG. 3A illustrates crumbs of bioabsorbable, implantable material that contains antibiotics.

FIG. 3B illustrates a crumb of bioabsorbable implantable material impregnated with antibiotics.

FIGS. 4A-4C shows orthopedic implants and geometric structures made from bioabsorbable implantable materials impregnated with antibiotics.

### DETAILED DESCRIPTION OF THE INVENTION

In one embodiment, a bioabsorbable, implantable material is impregnated (or fortified) with antibiotics and is rolled into a flexible sheet. During surgery, the sheet is cut to appropriate size and wrapped or formed about an orthopedic implant or any other part inside a body. As will be understood by those skilled in the art, the flexible sheet may be wrapped about a bone, a tissue or be placed in any part inside a body. The antibiotic elutes out from the sheet into the wound over a period of time to create a local bactericidal level of antibiotics. Thus, the sheet operates as a localized drug delivery system with minimal volume effect. As the sheet is formed from a bioabsorbable material, the sheet is eventually absorbed by the body.

In one embodiment, a bioabsorbable, implantable material is impregnated with crystalline antibiotics and rolled into a thin flexible sheet. A flexible laminate is then made by bonding a plurality of the sheets (or layers). Depending on the size of the antibiotics crystals and the number of layers in the flexible laminate, the antibiotics elute out into the wound over a desired period of time. Accordingly, the flexible laminate having a plurality of layers can operate as a drug delivery system for localized delivery of drugs over a period of time. In another embodiment, the antibiotics may be in a non-crystalline or a non-crystalline form such as a powder or any other suitable form. In another embodiment, a laminate can be made by bonding one or more layers that contain antibiotics and one or more layers that do not contain antibiotics.

In one embodiment, a method for localized delivery of drug about an implant, bone, tissue or any other part inside a body includes wrapping (or forming about) the implant, bone, tissue or the desired part with one or more sheets of antibiotics impregnated, bioabsorbable material.

As discussed before, perioperative infections in orthopedic implants frequently require a patient to undergo a subsequent operation to remove the implant and to sterilize the wound. The sheet is intended to reduce the potential for perioperative infection and also prevent the increase in costs resulting from additional procedures necessary to treat the infection.

In one embodiment, a bioabsorbable, implantable plastic is impregnated with antibiotics and formed into sheets, cellophanes or granules. Implantable plastics such as polyethylenes and polyetheretherketones can be impregnated with antibiotics and formed into sheets, cellophanes or granules. Also implantable plastics impregnated with antibiotics can be used to form various structures. Also, bioabsorbable polylactic acid (PLA), polyglycolic acid (PGA) and their copolymers can be impregnated with antibiotics and formed into sheets, cellophanes or granules. The sheets of cellophane can be cut to appropriate size and shaped about an implant or any other part inside a body, thus delivering antibiotics to a desired area with minimal volume affect. The antibiotics can be in a crystalline or a non-crystalline form.

In one embodiment, the drug delivery system comprises a flexible laminate made by bonding a plurality of layers of antibiotics impregnated, bioabsorbable cellophane. As will be understood by those skilled in the art, the flexible laminate can be made from any suitable material and just not limited to cellophane or plastics. Also, as will be understood by those skilled in the art, various drugs, besides antibiotics, can be delivered using the flexible laminate. Thus, the drug delivery system may deliver any suitable drugs and is not limited to the delivery of antibiotics. As will be understood by those skilled in the art, the antibiotics may be in a crystalline or a non-crystalline form. In another embodiment, the flexible laminate may include one or more layers that contain antibiotics and one or more layers that do not contain antibiotics.

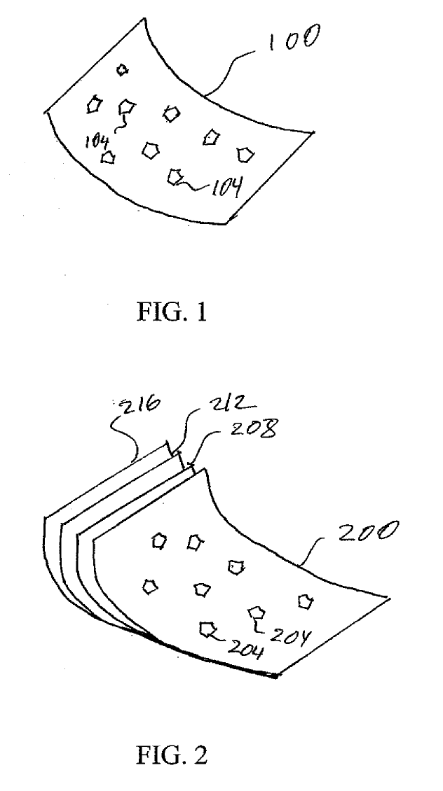

FIG. 1 illustrates a sheet 100 of bioabsorbable, implantable material that is impregnated with antibiotics 104. FIG. 2 illustrates a laminate 200 of bioabsorbable, implantable material that is impregnated with antibiotics 204. Individual layers 208-216 in the laminate can be impregnated with selected types of antibiotics or combination of antibiotics. Thus, individual layers in the laminate can have different types of drugs. Also, individual layers in the laminate can have different absorption characteristics so that different types of drugs can be delivered in controlled manner over a period of time. It will be appreciated that the laminate 200 can be formed by bonding any number of layers.

The approximate thickness of a laminate can be from hundredths of a millimeter to tenths of a millimeter. It will be appreciated by those skilled in the art that laminates with other thickness can be produced. Antibiotic crystals can have various sizes (e.g., micron or submicron magnitude). The laminate may also be fortified with dissolved antibiotics.

In one embodiment, a method for localized delivery of drug near or about an implant or any other part inside a body includes wrapping (or forming about) the implant (or any other part inside the body) with one or more sheets of antibiotics impregnated, bioabsorbable plastic. In another embodiment, a method for localized delivery of drug about an implant or any other part inside a body includes wrapping (or forming about) the implant (or any other part inside the body) with one or more bioabsorbable, antibiotics impregnated cellophanes.

More specifically, cellophane laminates containing one or more types of appropriate antibiotics are formed, thereby creating a multiple layered structure of bioabsorbable film. During or prior to surgery, these layered materials are cut into appropriate size and wrapped around orthopedic implants for insertion. The thin size of the bioabsorbable cellophanes or films do not impede the process of implantation. Once implanted in the body, the predetermined bioabsorbable degradation of the carrier material allows the delivery of antibiotics over a predetermined period of time to a localized environment. Materials such as polylactate and polyglycolactates have been used in orthopedic implants and have shown to cause no ill effects to the operative wound and they do not inhibit the healing process.

In one embodiment, the sheets of antibiotics impregnated, bioabsorbable materials (e.g., bioabsorbable plastic) can be used with dental, vascular and other implants. The sheets may be impregnated with other suitable drugs in addition to antibiotics.

As discussed before, when used intra-venusly (IV), antibiotics penetrate hard tissue relatively poorly. The drug delivery system comprising sheets of bioabsorbable material allow localized delivery of antibiotics directly to the hard tissue to increase the penetration rate of the antibiotics. As a result, the drug delivery system using sheets of bioabsorbable material decreases perioperative infection rate.

In one embodiment, a drug delivery system comprises a flexible laminate of multilayered, bioabsorbable material, wherein at least one layer is impregnated with a combination of two or more crystalline antibiotics. For example, a combination of Vancomycin and Tobramycin, which is known to be locally effective against Staphylococcal and Streptococcal species, can be delivered using the flexible laminate. In another embodiment, each layer of an implantable, bioabsorbable material can be impregnated with a particular type of antibiotics or a particular combination of antibiotics. For example, the first layer can be impregnated with a first type of antibiotic and the second layer can be impregnated with a second type of antibiotics.

The amount of time over which the antibiotics elute out, also referred to herein as the absorption time, can be increased by increasing the number of layers in the flexibleinate and also by increasing the size of the crystalline particles of antibiotics. The absorption time, accordingly, can be decreased by decreasing the number of layers in the laminate and also by decreasing the size of the crystalline antibiotics.

For example, the critical time for inhibition of bacteria delivered inadvertently to the wound during a surgery may be two to five days. The number of layers in the laminate and the size of the crystalline antibiotics can be adjusted appropriately so that the antibiotics in combination or alone (e.g., Tobramycin, Vancomycin or a combination of the two) elute out to the wound over a period of two to five days.

Many antibiotics, including Tobramycin and Vancomycin, are stable up to 100 degrees centigrade. Thus, these antibiotics are suitable for use in bioabsorbable sheets (e.g., cellophane) as they will be able to withstand the fabrication process.

In another embodiment, implantable, bioabsorbable material is used as a carrier for antibiotics. In one embodiment, implantable, bioabsorbable material is impregnated with antibiotics and fabricated as crumbs. The antibiotics may be in a crystalline or any other suitable form. The antibiotics impregnated crumbs are then introduced to the spaces of the wound for local delivery of antibiotics. The antibiotics impregnated crumbs may be used in an initial surgery for localized delivery of antibiotics to reduce the possibility of perioperative infection or during a surgery to treat perioperative infection.

For example, a patient with a perioperative infection can be returned to the operating room where the wound is washed out. The antibiotic impregnated crumbs are then introduced into the spaces of the wound. The antibiotics elutes over a number of days determined by the characteristics of the crumbs and the crystalline antibiotics. The crumbs need not be removed as they are known to be osteoconductive. In a spinal surgery, the antibiotics impregnated crumbs can also promote healing and fusion of the spine. Accordingly, the dual aim of delivering high dose of local antibiotics and achieving spinal fusion can be met without a return visit to the operating room.

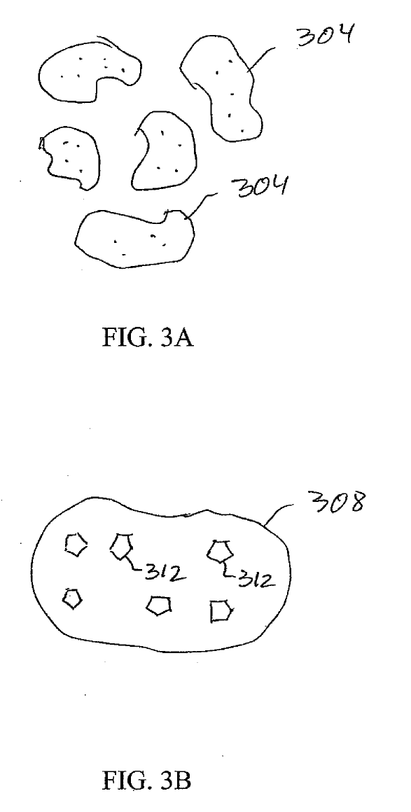

FIG. 3A illustrates crumbs 304 of bioabsorbable, implantable material that contains antibiotics. FIG. 3B illustrates a crumb 308 of bioabsorbable implantable material that is impregnated with antibiotics 312.

In one embodiment, one or more bone morphogenic proteins are added to the bioabsorbable, implantable material impregnated with antibiotics. The material is then formed into sheets, cellophanes or granules. The bioabsorbable material may be implantable plastics, carbon or other suitable materials.

As will be appreciated by those skilled in the art, biomorphogenic proteins are a class of biologics, which are used to initiate bony fusion and enhance the quality of bony fusion. The addition of the biomorphogenic protein to the bioabsorbable, implantable material can incite bone formation.

In one embodiment, a method for localized delivery of drug about an implant (or any other part inside a body) includes introducing antibiotics impregnated, bioabsorbable crumbs to spaces about the implant.

In another embodiment, orthopedic implants made from bioabsorbable materials can be impregnated with antibiotics. The implants impregnated with antibiotics will provide controlled delivery of antibiotics over a period of time.

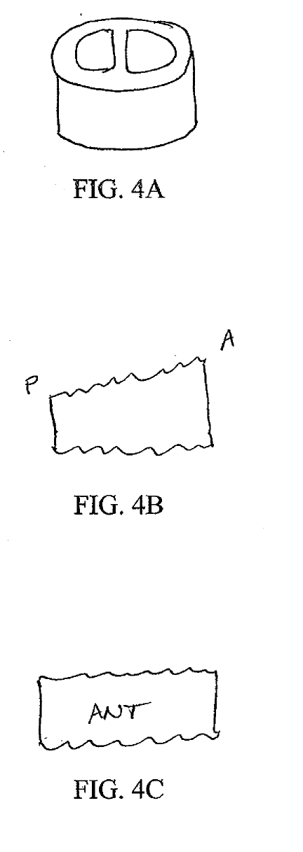

In one embodiment, bioabsorbable implantable materials impregnated with antibiotics can be used as osteoconductive scaffolding for bone formation. Antibiotics containing bioabsorbable implantable structures and geometric shapes can be used as substitutes for vertebral bodies or disk anteriorly where antibiotics will be delivered in high concentration to prevent infection and for eventual bone healing. FIGS. 4A, 4B and 4C shows various orthopedic implants and geometric structures made from bioabsorbable implantable materials impregnated with antibiotics.

While the compositions, structures, apparatus and methods of this invention have been described in terms of preferred embodiments, it will be apparent to those of skill in the art that variations may be applied to the compositions, structures, apparatus and/or methods and in the steps or in the sequence of steps of the method described herein without departing from the concept, spirit and scope of the invention. All such substitutes and modifications apparent to those skilled in the art are deemed to be within the spirit, scope and concept of the invention as defined by the appended claims.

What is claimed is:

1. A bioabsorbable, implantable, flexible sheet of material containing a predetermined quantity of one or more selected antibiotics, the sheet of material adapted to be placed inside a body for delivery of antibiotics.

2. The flexible sheet of material of claim 1, wherein the material is an implantable plastic.

3. The flexible sheet of material of claim 1, wherein the antibiotics is in a crystalline form.

4. The flexible sheet of material of claim 1, wherein the antibiotics is in a non-crystalline form.

5. The flexible sheet of material of claim 1, wherein the sheet of material is adapted to be wrapped about an implant.

6. The flexible sheet of material of claim 1, further comprising at least one bone morphogenic protein.

7. A bioabsorbable, implantable, flexible sheet of cellophane containing a predetermined quantity of one or more selected antibiotics, the sheet of cellophane adapted to be placed inside a body for delivery of antibiotics.

8. The flexible sheet of cellophane of claim 7, wherein the antibiotics is in a crystalline form.

9. The flexible sheet of cellophane of claim 7, wherein the antibiotics is in a non-crystalline form.

10. The flexible sheet of cellophane of claim 7, wherein the sheet of cellophane is adapted to be wrapped about an implant.

11. A flexible laminate made by bonding two or more layers of bioabsorbable, implantable material, at least one of the layers containing a predetermined quantity of one or more selected antibiotics, the flexible laminate adapted to be placed inside a body for delivery of antibiotics.

12. The flexible laminate of claim 11, wherein at least one of the layers is an implantable plastic.

13. The flexible laminate of claim 11, wherein at least one of the layers is an implantable cellophane.

14. The flexible laminate of claim 11, wherein the antibiotics is in a crystalline form.

15. The flexible laminate of claim 11, wherein the antibiotics is in a non-crystalline form.

16. The flexible laminate of claim 11, wherein the flexible laminate is adapted to be wrapped about an implant.

17. The flexible laminate of claim 11, her comprising at least one bone morphogenic proteins.

18. A method for delivery of drug, comprising the step of placing a bioabsorbable, implantable, flexible sheet of material containing a predetermined quantity of one or more selected antibiotics inside a body for delivery of antibiotics.

19. The method of claim 18, wherein the flexible sheet of material is an implantable plastic.

20. A method for delivery of drug, comprising the step of placing a flexible laminate inside a body, the flexible laminate made by bonding two or more layers of bioabsorbable, implantable material, at least one of the layers containing a predetermined quantity of one or more selected antibiotics.

21. The method of claim 20, further comprising the step of placing the laminate about an implant.

22. The method of claim 20, wherein at least one of the layers contain at least one biomorphogenic protein.
